(19) United States
(12) Patent Application Publication
(10) Pub. No.: US 2023/0400466 A1
(43) Pub. Date: Dec. 14, 2023

(54) METHODS AND SYSTEMS FOR RISK STRATIFICATION AND MANAGEMENT OF BLADDER CANCER

(71) Applicant: University of Houston System, Houston, TX (US)

(72) Inventors: Chandra Mohan, Houston, TX (US); Kamala Vanarsa, Houston, TX (US)

(21) Appl. No.: 18/324,107

(22) Filed: May 25, 2023

Related U.S. Application Data
(60) Provisional application No. 63/365,324, filed on May 25, 2022.

(51) Int. Cl. G01N 33/574; G01N 33/543

(52) U.S. Cl. CPC G01N 33/57407; G01N 33/54388; G01N 33/57488; G01N 2333/745; G01N 2333/96494

(57) ABSTRACT

Non-invasive methods of risk stratification and treatment of bladder cancer in a subject are based on evaluating D-dimer in a urine sample of the subject, and assay systems thereof for evaluation of D-dimer in the urine sample.

### CROSS-REFERENCE TO RELATED APPLICATIONS

This application claims the benefit of and priority to U.S. Provisional Application No. 63/365,324, filed on May 25, 2022, which is incorporated by reference herein in its entirety.

### TECHNICAL FIELD

The disclosure relates to systems and methods for evaluating peptides and their fragments in a biological sample for management of bladder cancer.

### BACKGROUND

Bladder cancer is a common malignancy in women and is the fourth most common malignancy in men. Bladder cancer ranges from unaggressive and usually noninvasive tumors that recur and commit patients to long-term invasive surveillance, to aggressive and invasive tumors with high disease-specific mortality. Seventy-five (75) % of BC patients present with non-muscle invasive bladder cancer (NMIBC) and are treated surgically or with BCG therapy. Of note, about 80% of these patients will have at least one recurrence, and about 30% will experience disease progression to muscle invasive bladder cancer (MIBC). Hence, patients initially diagnosed with and treated for BC are followed-up every 3-6 months (and then annually) in order to determine whether they have bladder cancer recurrence. At these follow-ups, the techniques commonly used to detect possible recurrence are cystoscopy and cytology (and sometime imaging). Unfortunately, currently available recurrence detection methods, including these tests, have subpar diagnostic capability, are expensive, and/or are invasive, with attendant side-effects. Indeed, the intensive follow-up (surveillance) program to detect bladder cancer recurrence using cystoscopy and urine cytology represents the most expensive follow-up program in oncology.

Shortcomings of cystoscopy, urine cytology, and other tests include one or more of the following. Cystoscopy with biopsy or transurethral resection is necessary for histological confirmation. However, cystoscopy is invasive, and relatively costly. Further, cystoscopy is often associated with complications including pain, urinary tract infection, and hematuria. Urine cytology is also commonly used for the diagnosis and surveillance of BC. This non-invasive method involves the examination of cells collected from a urine specimen. While urine cytology is non-invasive, it requires a specialized uropathologist and suffers from subjectiveness when grading urothelial carcinoma on urine samples thus resulting in considerable inter- and intra-observer variability even amongst cytopathologists. Although cytology has reasonably good specificity (about 86%), it exhibits low sensitivity of approximately 48%, which plummets to about 16% for low-grade tumors. Finally, neither tests are provided at point of care. Other tests that are feasible for primary care settings include the urine dipstick or microscopic urinalysis to detect hematuria, but these are not sensitive enough, and are of low specificity. Hence, there is a need for improved methods (e.g., biomarkers) for non-invasive detection of BC to support choice of treatment regimen for the patient.

### SUMMARY

The present disclosure provides methods for risk stratification or management of recurrent or new bladder cancer in patients by evaluating urine D-dimer levels. In certain embodiments, the D-dimer can be assayed by ELISA (enzyme-linked immunosorbent assay) or LFA (lateral flow assay). In the methods provided herein, in response to a urine test being positive for D-dimer, the patient is subject to a biopsy (via a cystoscopy) to confirm the diagnosis. If the urine test is negative for D-dimer, the patient will be reviewed at a subsequent follow-up visit. These methods provide a majority of patients with an alternative to unnecessary invasive or expensive procedures with suboptimal diagnostic profiles, leading to savings for the patient and overall healthcare industry, and reduction of incidence of cystoscopy-related side-effects. In an embodiment, the products and methods involve point-of-care lateral flow test strips (LFT) for evaluation of urine D-dimer that can be used by the patient to self-test the urine for bladder cancer recurrence at home periodically (e.g., every month, every 2 weeks). In contrast, under the current protocols, cancer recurrence may be detected only at the next follow up visit, which could be 6 months or a year later. The products and methods herein provide for home-based surveillance for bladder cancer incidence or recurrence. Recurrences of bladder cancer are detected earlier than it is currently being detected, potentially leading to decreased patient morbidity and mortality.

Certain embodiments include methods for risk stratification for bladder cancer in a subject. One such method includes the steps of evaluating a level of D-dimer in a first urine sample obtained from the subject as compared to a predetermined threshold level, and in response to the level of D-dimer in the first urine sample being above the predetermined threshold, identifying the subject as at an increased risk of having bladder cancer and as a candidate for an invasive testing for bladder cancer. Furthermore, this method includes the step of identifying the subject as not at an increased risk of having bladder cancer and as a candidate for not having an invasive testing for bladder cancer in response to the level of D-dimer in the first urine sample being at or below the predetermined threshold,. In certain embodiments, the predetermined threshold is based on a Receiver Operating Curve Area Under Curve (ROC-AUC) analysis. In certain embodiments, the predetermined threshold for test positivity is about 90.3 pg/ml. Urine levels of D-dimer in other diseases (such as urinary tract infections) can be determined. Certain embodiments are directed to methods for risk stratification for bladder cancer recurrence in a subject who has had bladder cancer.

In certain embodiments, the method further includes the following steps: (i) in response to the level of D-dimer in the first urine sample being at or below the predetermined threshold, assessing a level of D-dimer in a second urine sample obtained from the subject as compared to a predetermined threshold, wherein the second urine sample is obtained at two or more weeks after the time the first urine sample was obtained from the subject; (ii) in response to the level of D-dimer in the second urine sample being above the predetermined threshold, identifying the subject as at an increased risk of having bladder cancer and as a candidate for an invasive testing for bladder cancer; and (iii) in response to the level of D-dimer in the second urine sample being at or below the predetermined threshold, identifying the subject as not at an increased risk of having bladder cancer and as a candidate for not having an invasive testing for bladder cancer.

In certain embodiments, the invasive testing for bladder cancer includes one or more of cystoscopy, biopsy, and transurethral resection. In certain embodiments, the method further includes the step of identifying the subject as being at an increased risk of having bladder cancer as a candidate for urine cytology or nuclear matrix protein 22 analysis in a urine sample.

In certain embodiments, the method further includes the steps of assessing an elevated level of one or more of MMP-1, Apolipoprotein A1, Proteinase 3, and Apolipoprotein L1 in the urine sample obtained from the subject as compared to predetermined threshold levels of MMP-1, Apolipoprotein A1, Proteinase 3, or Apolipoprotein L1, and determining that the subject is at increased risk of having bladder cancer based on the elevated levels of the one or more of MMP-1, Apolipoprotein A1, Proteinase 3, and Apolipoprotein L1 in the urine sample.

In certain embodiments, the method further includes the steps of assessing an elevated level of one or more of Apolipoprotein A1 and IL-8 in the urine sample obtained from the subject as compared to predetermined threshold levels of Apolipoprotein A1 or IL-8; and determining that the subject is at increased risk of having bladder cancer based on the elevated levels of the one or more of Apolipoprotein A1 and IL-8 in the urine sample.

Certain embodiments of a method for identifying a subject for invasive testing for bladder cancer includes the steps of assessing an elevated level of D-dimer in a urine sample obtained from the subject as compared to a predetermined threshold level, determining that the subject is at risk of having bladder cancer based on the elevated level of D-dimer in the urine sample, and performing an invasive testing for bladder cancer on the subject. In certain embodiments, the subject has had bladder cancer and is being evaluated for bladder cancer recurrence. In certain embodiments, the invasive testing for bladder cancer includes one or more of cystoscopy, biopsy, and transurethral resection.

In certain embodiments, the method further includes conducting urine cytology or detecting a level of nuclear matrix protein 22 in a urine sample obtained from the subject at an increased risk of having bladder cancer. In certain embodiments, the method further includes the step of assessing an elevated level of one or more of MMP-1, Apolipoprotein A1, Proteinase 3, and Apolipoprotein L1 in the urine sample obtained from the subject as compared to predetermined threshold levels of MMP-1, Apolipoprotein A1, Proteinase 3, or Apolipoprotein L1, and determining that the subject is at increased risk of having bladder cancer based on the elevated levels of the one or more of MMP-1, Apolipoprotein A1, Proteinase 3, and Apolipoprotein L1in the urine sample. In certain embodiments, the method further includes the steps of assessing an elevated level of one or more of Apolipoprotein A1 and IL-8 in the urine sample obtained from the subject as compared to predetermined threshold levels of Apolipoprotein A1 or IL-8; and determining that the subject is at increased risk of having bladder cancer based on the elevated levels of the one or more of Apolipoprotein A1 and IL-8 in the urine sample.

Certain embodiments of a method for treating a subject for bladder cancer includes the following steps: (i) assessing an elevated level of D-dimer in a urine sample obtained from the subject as compared to a predetermined threshold level through an enzyme-linked immunosorbent assay (ELISA) or a lateral flow assay (LFA) or a vertical flow assay (VFA), (ii) in response to the level of D-dimer in the urine sample being above the predetermined threshold, identifying the subject as at increased risk of having bladder cancer, (iii) performing an invasive testing for bladder cancer on the subject, (iv) determining that the subject has bladder cancer based on results of the invasive testing, and (v) treating the subject for the bladder cancer by one or more of surgical resection, radiation, chemotherapy, and hormone therapy. These therapeutic modalities can include immune-checkpoint inhibitors, monoclonal antibodies, as well as intravesical or systemic chemotherapy such as cisplatin, fluorouracil, mitomycin, gemcitabine, methotrexate, vinblastine, doxorubicin/Adriamycin and paclitaxel.

In certain embodiments, the subject has had bladder cancer, and the treatment is for bladder cancer recurrence. Methods and products disclosed here are used as prognostic markers. Evaluation of the Urine D-dimer may also be used to track response to treatment, following any of the previously treatment modalities. In certain embodiments, the invasive testing for bladder cancer includes one or more of cystoscopy, biopsy followed by pathological examination, transurethral resection followed by pathological examination, urine cytology, and detecting a level of nuclear matrix protein 22 in a urine sample.

Certain embodiments include a product for detecting a level of D-dimer in a urine sample for risk stratification for bladder cancer. The product is configured to accommodate an ELISA or LFA or VFA. In certain embodiments, the assay system includes a lateral flow test strip. In certain embodiments, the product can also be used to detect levels of one or more of MMP-1, Apolipoprotein A1, Proteinase 3, and Apolipoprotein L1 in the urine sample. In certain embodiments, the product can be used to detect levels of Apolipoprotein A1 or IL-8.

In certain embodiments, the assay system includes a kit containing one or more of the following: a capture reagent and a detection reagent that each bind to D-dimer, a signal development element that can bind to the detection reagent, a washing buffer, a blocking buffer, information for a calibration curve to relate a signal to the level of D-dimer, and/or an instruction for use. In other embodiments, these same reagents may be configured to detect urine D-dimer using either an LFA or VFA point-of-care test format, for use in a primary care setting, at home (i.e., self-testing by the patient), or in an outpatient setting, with semi-quantitative or quantitative readouts.

Aspects and advantages of these exemplary embodiments and other embodiments are discussed in detail herein. Moreover, both the foregoing information and the following detailed description provide illustrative examples of various aspects and embodiments. The features of the various embodiments described herein are not mutually exclusive and may exist in various combinations and permutations. This Summary section is not intended to identify key features or essential features of the claimed subject matter, nor is it intended to be used as an aid in determining the scope of the claimed subject matter. The claimed subject matter is not limited to implementations that solve any or all disadvantages noted in the Background section.

### BRIEF DESCRIPTION OF THE DRAWINGS

The accompanying drawings, which are included to provide a further understanding of the embodiments of the present disclosure, are incorporated in and constitute a part of this specification, illustrate embodiments of the present disclosure, and together with the detailed description, serve to explain principles of the embodiments discussed herein. No attempt is made to show structural details of this disclosure in more detail than may be necessary for a fundamental understanding of the embodiments discussed herein and the various ways in which they may be practiced. According to common practice, the various features of the drawings discussed below are not necessarily drawn to scale. Dimensions of various features and elements in the drawings may be expanded or reduced to more clearly illustrate embodiments of the disclosure.

FIG. 1 is a diagrammatic representation of a method for risk stratification or treating for bladder cancer, according to certain embodiments of the present disclosure.

FIG. 2A is a dot plot of urine D-dimer levels without creatinine normalization. Tested samples include 31 urology clinic controls (UC) and 37 bladder cancer patients (BC). Creatinine un-normalized urine protein levels are displayed. UC is represented by a circle and BC is represented by a square. The asterisks indicate the level of significance between the groups: ****p<0.0001.

FIG. 2B depicts ROC-AUC plots generated for urine D-dimer in its ability to discriminate BC from UC, without creatine normalization. AUC values and p-values are listed. An AUC close to 1 indicates that the protein has a higher discriminatory potential to distinguish between the two groups.

FIG. 3 depicts a dot plot of urine D-dimer in a second validation cohort of Chinese ethnicity, containing 91 BC patients and 77 UC patients. The UC subjects included 18 with kidney cancer, 50 with renal tract stones, and the rest with renal cyst, hamartoma, or other non-BC conditions. D-dimer values normalized by creatinine are shown for each group. UC is represented by a circle and BC is represented by a square. The asterisks indicate the level of significance between the groups: **p<0.01.

### DETAILED DESCRIPTION

The description may use the phrases “in certain embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous. The term “about” or “approximately” are defined as being close to as understood by one of ordinary skill in the art. In one non-limiting embodiment, the terms are defined to be within 10%, preferably within 5%, more preferably within 1%, and most preferably within 0.5%. The terms “reducing,” “reduced,” or any variation thereof, when used in the claims and/or the specification includes any measurable decrease of one or more components in a mixture to achieve a desired result. The terms “wt. %”, “vol. %”, or “mol. %” refers to a weight, volume, or molar percentage of a component, respectively, based on the total weight, the total volume of material, or total moles, that includes the component. In a non-limiting example, 10 grams of component in 100 grams of the material is 10 wt. % of component.

As used herein, a “subject” is an animal, such as a mammal, including a primate (such as a human, a non-human primate, e.g., a monkey). In certain aspects, the subject is a human, who has, is suspected of having, at risk of developing, or has had bladder cancer. The subject can be an adult subject or a pediatric subject.

As used herein, “diagnosis” refers to methods by which one skilled in the art can evaluate or determine the probability (or a likelihood) of whether or not a patient has a certain disease or is at risk of developing the disease. One such method includes using the results of an assay, for example an immunoassay, to detect a level of D-dimer in a sample obtained from a subject, optionally together with other clinical characteristics, to arrive at a diagnosis (the occurrence or nonoccurrence) of bladder cancer in the subject. “Diagnosis”, “diagnose”, or making a diagnosis does not necessarily mean that there is a complete accuracy of the assessment for the disease. A skilled clinician can use biomarker results together with other clinical indicia to arrive at a diagnosis. A measured biomarker (e.g., D-dimer) level on one side of a predetermined diagnostic threshold (e.g., higher than the threshold) indicates a greater likelihood of the occurrence of disease (e.g., bladder cancer) in the subject relative to a measured level on the other side of (e.g., lower than) the predetermined diagnostic threshold.

As used herein, “risk stratification” refers to methods used to estimate or determine an increased risk of a certain disease in a subject, or a population of subjects, as compared to a healthy or a non-diseased population. The present disclosure provides for non-invasive methods for risk stratification for bladder cancer in a subject. In certain embodiments, the subject has had bladder cancer, and the risk stratification is for bladder cancer recurrence.

MIBC is a more advanced stage of bladder cancer. MIBC is when the cancer has grown far into the wall of the bladder (Stages T2 and beyond). MIBC is often treated with a combination of one or more of cystectomy, transurethral resection of bladder tumor (TURBT), chemotherapy, and radiation. MIBC has high rate of recurrence. For example, in patients who have a cystectomy, the recurrence rate can be from 20-30% for stage T2, 40% for T3, greater than 50% for T4, and often higher when lymph nodes are involved.

According to the TMN system (American Joint Committee on Cancer. Urinary Bladder. Amin M B et al, eds. AJCC Cancer Staging Manual. 8th ed. New York: Springer; 2017), primary tumor of bladder cancer can be classified as set forth in Table 1.

TABLE 1
Primary tumor (T) classification for bladder cancer

| | |
|---|---|
| TX | Primary tumor cannot be assessed |
| T0 | No evidence of primary tumor |
| Ta | Noninvasive papillary carcinoma |
| Tis | Carcinoma in situ: “flat tumor” |
| T1 | Tumor invades lamina propria (subepithelial connective tissue) |
| T2 | Tumor invades muscularis propria |
| pT2a | Tumor invades superficial muscularis propria (inner half) |
| pT2b | Tumor invades deep muscularis propria (outer half) |
| T3 | Tumor invades perivesical tissue |
| pT3a | Microscopically |
| pT3b | Macroscopically (extravesical mass) |
| T4 | Tumor invades any of the following: prostatic stroma, seminal |
| | vesicles, uterus, vagina, pelvic wall, abdominal wall |
| T4a | Tumor invades prostatic stroma, uterus, vagina |
| T4b | Tumor invades pelvic wall, abdominal wall |

D-dimer is a soluble degradation product of fibrin produced during the fibrinolytic process. Products and methods described herein are directed to assessing a level of D-dimer in a urine sample for risk stratification for bladder cancer. In certain embodiments, a level of D-dimer in a urine sample is detected by an immunoassay. Immunoassays generally involve contacting a sample with at least one antibody that specifically binds to D-dimer or another biomarker of interest. A signal is then generated indicative of the presence or amount of complexes formed by the binding of the biomarker or fragment thereof in the sample to the antibody. The signal is then related to the presence or amount of the biomarker (e.g., D-dimer) in the sample. Numerous methods and devices are well known for the detection and analysis of biomarkers. See, e.g., The Immunoassay Handbook, David Wild, ed. Stockton Press, New York, 1994, which is hereby incorporated by reference in its entirety.

Any suitable immunoassay may be utilized, for example, enzyme-linked immunoassays (ELISA), lateral flow assay (LFA), radioimmunoassays (RIAs), and competitive binding assays. The assay devices and methods known in the art can utilize labeled molecules in various sandwich, competitive, or non-competitive assay formats, to generate a signal that is related to the presence or amount of the biomarker of interest. Suitable assay formats also include chromatographic, mass spectrographic, and protein “blotting” methods. Additionally, certain methods and devices, such as biosensors and optical immunoassays, may be employed to determine the presence or amount of D-dimer without the need for a labeled molecule.

In certain embodiments, the assay system is an enzyme-linked immunosorbent assay (ELISA) system. ELISA, used interchangeably with enzyme immunoassay (EIA), is a solid phase-based assay technique designed for detecting and quantifying soluble substances such as peptides, proteins, antibodies, and hormones. In an ELISA, the antigen is immobilized on a solid surface and then complexed with an antibody that is linked to a reporter enzyme. Detection is accomplished by measuring the activity of the reporter enzyme via incubation with the appropriate substrate to produce a measurable product. The most crucial element of an ELISA is a highly specific antibody-antigen interaction.

Any format of ELISA can be used in accordance with the methods provided herein. Such format of ELISA includes direct, indirect, or sandwich capture and detection methods. The key step is immobilization of the antigen of interest, accomplished by either direct adsorption to the assay plate or indirectly via a capture antibody that has been attached to the plate. The antigen is then detected either directly (labeled primary antibody) or indirectly (such as labeled secondary antibody). A sandwich ELISA assay indirectly immobilizes and indirectly detects the presence of the target antigen, wherein the analyte to be measured is bound between two primary antibodies, each detecting a different epitope of the antigen—the capture antibody and the detection antibody. The direct detection method uses a primary antibody labeled with a reporter enzyme or a tag that reacts directly with the antigen. Direct detection can be performed with an antigen that is directly immobilized on the assay plate or with the capture assay format. Direct detection, while not widely used in ELISA, is quite common for immunohistochemical staining of tissues and cells. The indirect detection method uses a labeled secondary antibody or a biotin-streptavidin complex for amplification and is the most popular format for ELISA. The secondary antibody has specificity for the primary antibody. In a sandwich ELISA, it is critical that the secondary antibody is specific for the detection of the primary antibody only (and not the capture antibody) or the assay will not be specific for the antigen. Generally, this is achieved by using capture and primary antibodies from different host species (e.g., mouse IgG and rabbit IgG, respectively).

In certain embodiments, the assay system is a lateral flow assay (LFA) system. A lateral flow assay system is a form of immunoassay in which the test sample flows in a chromatographic fashion along a bibulous or non-bibulous porous solid substrate. Lateral flow tests can operate as either competitive or sandwich format assays.

Lateral flow devices can be disposable, single use test devices. For example, in certain embodiments, the LFA system includes a lateral flow test strip. A sample is applied to the test device at an application zone and transits the substrate, where it encounters lines or zones which have been pretreated with an antibody or antigen. The term “test zone” as used herein refers to a discrete location on a lateral flow test strip which is interrogated in order to generate a signal related to the presence or amount of an analyte of interest (e.g., D-dimer). The detectable signal may be read visually or obtained by inserting the disposable test device into an analytical instrument such as a reflectometer, a fluorometer, a transmission photometer, or any other instrument. Sample may be applied without pretreatment to the application zone, or may be premixed with one or more assay reagents prior to application. In the latter case, the antibody may be provided in a separate container from the disposable test device.

Point-of-care lateral flow test strips (LFT) for urine D-dimer can be used by the subject (e.g., patient) to self-test the urine for bladder cancer recurrence from the comfort of his/her home every month (or even every 2 weeks). This protocol for home-based surveillance for bladder cancer recurrence has an advantage of detecting bladder cancer recurrence earlier than using an invasive testing (e.g., cystoscopy) as the first line of risk stratification, leading to decreased patient morbidity and mortality. In certain embodiments, the product can also be used to detect levels of one or more of MMP-1, Apolipoprotein A1, Proteinase 3, and Apolipoprotein L1 in the urine sample. In certain embodiments, the product can be used to detect levels of Apolipoprotein A1 or IL-8.

In certain embodiments, the assay system includes: a capture reagent and a detection reagent that each bind to D-dimer, a signal development element that can bind to the detection reagent, a washing buffer, a blocking buffer, information for a calibration curve to relate a signal to the level of D-dimer, and/or an instruction for use.

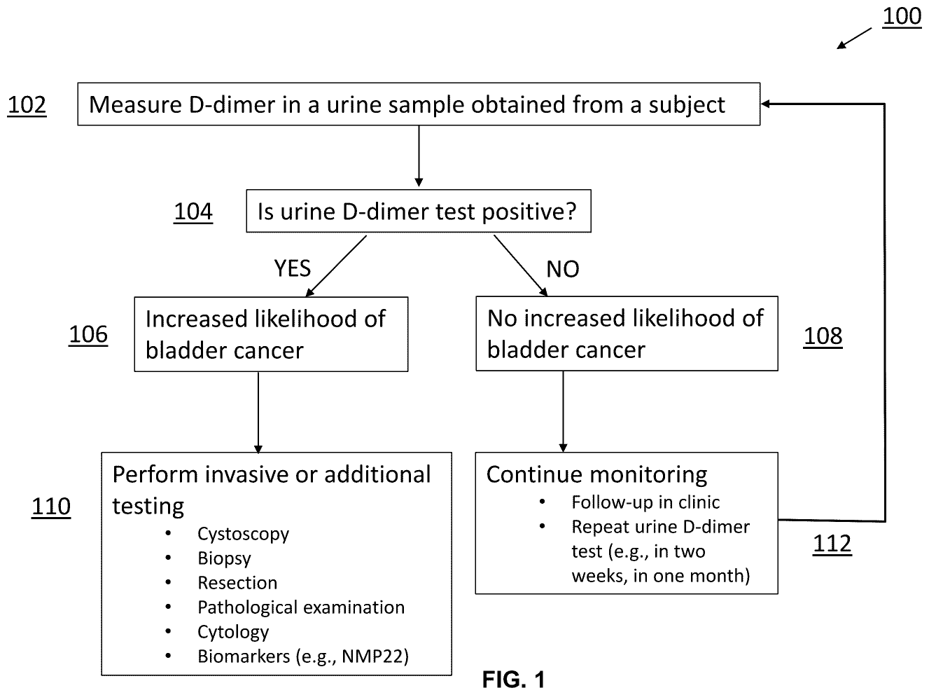

Certain embodiments include methods for risk stratification for bladder cancer in a subject. One such method includes the steps of detecting a level of D-dimer in a first urine sample obtained from the subject as compared to a predetermined threshold level, and in response to the level of D-dimer in the first urine sample being above the predetermined threshold, identifying the subject as at an increased risk of having bladder cancer and as a candidate for an invasive testing for bladder cancer; and in response to the level of D-dimer in the first urine sample being at or below the predetermined threshold, identifying the subject as not at an increased risk of having bladder cancer and as a candidate for not having an invasive testing for bladder cancer. An exemplary method 100 for risk stratification for bladder cancer is set forth in FIG. 1. According to methods provided herein, a urine sample is obtained from a subject (e.g., at risk of bladder cancer, at risk of bladder cancer recurrence). At 102, the method measures a level of D-dimer in the urine sample obtained from the subject. The method continues to 104, where the level of D-dimer in the sample is determined to be positive or negative. The level of D-dimer in the sample can be compared with a predetermined threshold. The urine D-dimer test can be considered “positive” when the level of D-dimer in the sample is above the predetermined threshold. The urine D-dimer test can be considered “negative” when the level of D-dimer in the sample is below the predetermined threshold.

In response to a urine D-dimer test being positive, at 106, the subject is identified as being at increased likelihood (e.g., risk) of having bladder cancer. At 108, the subject is further identified as a candidate for invasive or additional testing to confirm bladder cancer diagnosis. Invasive testing can include cystoscopy, biopsy, and resection, followed by pathological examination of samples obtained from the subject. Additional testing can include urine cytology and measurement of a biomarker (e.g., NMP22).

In response to a urine D-dimer test being negative, at 108, the subject is identified as not being at increased likelihood (e.g., risk) of having bladder cancer. At 112, the subject is further identified as a candidate for continued monitoring, e.g., without undergoing an invasive testing. Continued monitoring can include follow up clinic visits and checkup and repeating urine D-dimer test, e.g., the steps of 102, 104, 106, 110, 108, and/or 112, at two weeks or more (e.g., 2, 3, 4, 5, 6, 7, 8, 9, 10, 11, or 12 weeks or longer) after the first urine D-dimer test.

Selecting a predetermined threshold, e.g., to compare with a D-dimer level in a sample at 104 involves, among other things, consideration of the probability of disease, distribution of true and false diagnoses at different test thresholds, and estimates of the consequences of treatment (or a failure to treat) based on the diagnosis. For example, when considering administering a specific therapy, which is highly efficacious and has a low level of risk, few tests are needed because clinicians can accept substantial diagnostic uncertainty. On the other hand, in situations where treatment options are less effective and riskier, clinicians often need a higher degree of diagnostic certainty. Thus, cost/benefit analysis is involved in selecting a diagnostic threshold.

Suitable thresholds may be determined in a variety of ways. One method is setting a percentile (e.g., 97.5 percentile) of normal population. Another method may be to look at serial samples from the same patient, where a prior “baseline” result is used to monitor for temporal changes in a D-dimer level. And, another method involves setting a cut-off so as to maximize the ROC Youden index. The Youden Index provides the maximum potential effectiveness of the D-dimer.

In certain embodiments of the methods provided herein, the subject has had bladder cancer, and the risk stratification is for bladder cancer recurrence. In certain embodiments, the method further includes the following steps: (i) in response to the level of D-dimer in the first urine sample being at or below the predetermined threshold, assessing a level of D-dimer in a second urine sample obtained from the subject as compared to a predetermined threshold, wherein the second urine sample is obtained at two or more weeks after the time the first urine sample was obtained from the subject; (ii) in response to the level of D-dimer in the second urine sample being above the predetermined threshold, identifying the subject as at an increased risk of having bladder cancer and as a candidate for an invasive testing for bladder cancer; and (iii) in response to the level of D-dimer in the second urine sample being at or below the predetermined threshold, identifying the subject as not at an increased risk of having bladder cancer and as a candidate for not having an invasive testing for bladder cancer.

In certain embodiments, the invasive testing for bladder cancer includes one or more of cystoscopy, biopsy, and transurethral resection.

In certain embodiments, the D-dimer test as provided herein is used in combination with other risk stratification or diagnosis methods for bladder cancer (e.g., bladder cancer treatment). For example, the method can further comprise identifying the subject identified as being at an increased risk of having bladder cancer as a candidate for additional tests, e.g., urine cytology or nuclear matrix protein 22 detection in a urine sample. In certain embodiments, the method further includes the step of assessing an elevated level of one or more of MMP-1, Apolipoprotein A1, Proteinase 3, and Apolipoprotein L1 in the urine sample obtained from the subject as compared to predetermined threshold levels of MMP-1, Apolipoprotein A1, Proteinase 3, or Apolipoprotein L1, and determining that the subject is at increased risk of having bladder cancer based on the elevated levels of the one or more of MMP-1, Apolipoprotein A1, Proteinase 3, and Apolipoprotein L1 in the urine sample. In certain embodiments, the method further includes the steps of assessing an elevated level of one or more of Apolipoprotein A1 and IL-8 in the urine sample obtained from the subject as compared to predetermined threshold levels of Apolipoprotein A1 or IL-8; and determining that the subject is at increased risk of having bladder cancer based on the elevated levels of the one or more of Apolipoprotein A1 and IL-8 in the urine sample.

Cytology, nuclear matrix protein 22 (NMP22) kit, NMP22 BladderChek Test, BTA-TRAK/Stat, Cell Search, and UroVysion are FDA-approved for initial diagnosis and surveillance of BC, and can be used in conjunction with the D-dimer testing provided herein. The NMP22 test evaluates for nuclear matrix protein in the urine, with widely varying sensitivity and specificity metrics. In a meta-analysis of 19 studies, the sensitivity and specificity of NMP22 were 56% (52-59%) and 88% (87-89%), respectively, with an AUC of 0.83. The mean sensitivity of the NMP22 test for Ta, T1≥T2, Tis, G1, G2, and G3 disease was 13.7%, 29.5%, 74.0%, 34.6%, 44.2%, 56.3%, and 67.3%, respectively.

One study reported the sensitivities of the NMP22 test and cytology for detection of BC recurrence to be 78.8% and 44.2%, respectively, while the specificities were 69.6% and 83.7%, respectively (Hosseini et al. 2012 Urol J 9(1):367-72). Another study reported the specificities of the NMP22 test and cytology to be 77% and 96%, respectively, and combining the two tests increased sensitivity to 91% (Kumar et al. 2006 J Clin Oncol 36(3):172-175). Others have analyzed results from a multicenter study comparing both tests in 1272 subjects, of whom 6% had BC (Lotan et al. 2009 Br J Urol 103:1368-1374). In that study, although both NMP22 and cytology were independent predictors of BC, the area under curve (AUC) for NMP22 was 76% as compared to 56.2% for cytology. However, the NMP22 test has a low PPV and a low post-positive-test likelihood ratio compared to cytology (3.5 vs 19).

The risk stratification and detection methods provided herein can be combined with any treatment for bladder cancer to treat subjects identified as having bladder cancer. As used herein, the term “treating” or “treatment” in the context of treating a disease refers to a beneficial or desired result, such as reducing at least one associated sign, symptom, condition, or complication in a subject. “Treatment” also refers to a prophylactic treatment, such as prevention of a disease or prevention of at least one sign, symptom, condition, or complication associated with the disease. “Treatment” can also mean prolonging survival as compared to expected survival in the absence of treatment. Treatment of bladder cancer according to the methods provided herein can include surgical resection, chemotherapy, radiation, and hormone therapy.

Certain embodiments of a method for treating a subject for bladder cancer includes the following steps: (i) assessing an elevated level of D-dimer in a urine sample obtained from the subject as compared to a predetermined threshold level through an enzyme-linked immunosorbent assay (ELISA) or a lateral flow assay (LFA); (ii) in response to the level of D-dimer in the urine sample being above the predetermined threshold, identifying the subject as at increased risk of having bladder cancer; (iii) performing an invasive testing for bladder cancer on the subject; (iv) determining that the subject has bladder cancer based on results of the invasive testing; and (v) treating the subject for the bladder cancer by one or more of surgical resection, radiation, chemotherapy, and hormone therapy. In certain embodiments, the subject has had bladder cancer, and the treatment is for bladder cancer recurrence. In certain embodiments, the invasive testing for bladder cancer includes one or more of cystoscopy, biopsy followed by pathological examination, transurethral resection followed by pathological examination, urine cytology, and assessing a level of nuclear matrix protein 22 in a urine sample. In certain embodiments, the method further includes the step of assessing an elevated level of one or more of MMP-1, Apolipoprotein A1, Proteinase 3, and Apolipoprotein L1 in the urine sample obtained from the subject as compared to predetermined threshold levels of MMP-1, Apolipoprotein A1, Proteinase 3, or Apolipoprotein L1, and determining that the subject is at increased risk of having bladder cancer based on the elevated levels of the one or more of MMP-1, Apolipoprotein A1, Proteinase 3, and Apolipoprotein L1 in the urine sample. In certain embodiments, the method further includes the steps of assessing an elevated level of one or more of Apolipoprotein A1 and IL-8 in the urine sample obtained from the subject as compared to predetermined threshold levels of Apolipoprotein A1 or IL-8; and determining that the subject is at increased risk of having bladder cancer based on the elevated levels of the one or more of Apolipoprotein A1 and IL-8 in the urine sample.

The methods of risk stratification and detection of bladder cancer provided herein, using urine D-dimer for the surveillance of bladder cancer (e.g., recurrence), offers several advantages compared to the current technologies. In certain embodiments, the methods described herein are more accurate, with ROC AUC values of 96-97%, compared to current diagnostic approaches. These methods are non-invasive and pain-free with little to no side effects. In certain embodiments, the methods are easy to handle and provide quick assay results can be obtained within 10 minutes if assayed as a lateral flow test for detecting urine D-dimer. In certain embodiments, the methods described herein require minimal training or expertise as patients can self-test themselves using a lateral flow test strip, in contrast to cystoscopy and cytology, both of which need expensive instruments, dedicated trained expertise available only at urology referral centers. In certain embodiments, the methods described herein can potentially help detect bladder cancer recurrence earlier because patients can self-check their urine every week or two, in contrast to current follow up periods (which range from 3 months to >1 year). Earlier detection of bladder cancer recurrence will translate to earlier therapy, leading to reduced morbidity and mortality.

### EXAMPLES

### Example 1: Identification of D-Dimer as a Biomarker for Bladder Cancer

A comprehensive targeted proteomic interrogation of 1300 specific proteins in bladder cancer was conducted, comparing to urine protein profiles of urology clinic (UC) control patients. UC does not refer to urothelial carcinoma but for urology clinic controls. The top 30 most discriminatory proteins were further validated by ELISA. The independent validation cohort for ELISA consisted of 31 UC samples and 37 BC samples of different disease stages. Of these, 30 subjects (10 Ta samples, 10 Tis samples, 10 T1 samples) were classified as NMIBC while 7 subjects (stage T2-T4 samples) were classified as MIBC. The urology clinic controls included patients investigated for hematuria, but found not to have any urological cancers. Unless stated otherwise, all bladder cancer subjects included in this study had urothelial cancer. The results of this validation are shown in Table 2. Of the 30 most discriminatory proteins validated by ELISA, D-dimer was the urine protein with the best accuracy values for distinguishing BC patients from controls. Moreover, urine D-dimer exhibited sensitivity and specificity values of 0.90 to 0.95, far exceeding current diagnostic platforms.

TABLE 2
Urinary protein markers for detecting BC over UC validated by ELISA

| | UC | BC | | | | | | |
|---|---|---|---|---|---|---|---|---|
| | (mean/ | (mean/ | Fold | | | | | |
| Protein | median) | medium) | Change | Cut Off | AUC | Specificity | Sensitivity | Sensitivity^{0.8} |
| 14-3-3 sigma^{p} | 45.5 | 32.1 | 0.7 | 16.3 | 0.50 | 0.74 | 0.43 | 0.22 |
| | (12.2) | (12.9) | | | | | | |
| α2 | 10666.3 | 27085.6 | 2.5**** | 2241.9 | 0.78**** | 0.81 | 0.78 | 0.78 |
| Macroglobulinⁿ | (1032.9) | (9119.2) | | | | | | |
| α-synucleinⁿ | 1.8 | 4.0 | 2.2 | 0.9 | 0.55 | 0 . . . 74 | 0.49 | 0.32 |
| | (0.7) | (0.8) | | | | | | |
| Apolipoprotein | 3.1(0) | 247.1 | 79.1**** | 10.1 | 0.91**** | 0.97 | 0.84 | 0.86 |
| A1ⁿ | | (118.3) | | | | | | |
| Apolipoprotein | 94.9 | 615.8 | 6.5** | 24.5 | 0.72.*** | 0.65 | 0.73 | 0.54 |
| L1ⁿ | (13.7) | (184.9) | | | | | | |
| C2ⁿ | 12.7 | 523.1 | 4.1.2**** | 5.7 | 0.84**** | 0.81 | 0.81 | 0.81 |
| | (1) | (42.9) | | | | | | |
| Calgranulin Bⁿ | 13849.6 | 110696.2 | 0.8**** | 14139.2 | 0.85**** | 0.81 | 0.84 | 0.84 |
| | (2650.2) | (44584.8) | | | | | | |
| CLU^{p} | 183900.5 | 334394.9 | 1.8*** | 94372.3 | 0.74**** | 0.74 | 0.76 | 0.62 |
| | (55358.3) | (190605.7) | | | | | | |
| D-dimer^{p} | 100.6 | 13561.8 | 134.8**** | 278.1 | 0.96**** | 0.90 | 0.95 | 0.97 |
| | (1) | (4301.2) | | | | | | |
| Elastaseⁿ | 8.7 | 39.1 | 4.5**** | 3.8 | 0.79**** | 0.94 | 0.65 | 0.65 |
| | (0.1) | (5.7) | | | | | | |
| Endocan^{p} | 7.5 | 1134.7 | 151.8*** | 10.9 | 0.74**** | 0.90 | 0.60 | 0.62 |
| | (1) | (18.2) | | | | | | |
| Ficolin-3ⁿ | 346.4 | 376.7 | 1.1 | 191.2 | 0.56 | 0.81 | 0.46 | 0.46 |
| | (124.5) | (168.3) | | | | | | |
| Fibronectinⁿ | 807.9 | 1033.7 | 1.3*** | 99.2 | 0.77**** | 0.65 | 0.84 | 0.54 |
| | (49.2) | (386.9) | | | | | | |
| HCC-1^{p} | 78.3 | 3377.9 | 43.2*** | 1.8 | 0.71*** | 0.84 | 0.62 | 0.62 |
| | (0) | (9.5) | | | | | | |
| IgAⁿ | 532.3 | 2727.7 | 5.1**** | 312.7 | 0.85**** | 0.87 | 0.81 | 0.84 |
| | (117.6) | (1521.8) | | | | | | |
| IL-8^{p} | 499.3 | 365.1 | 7.4**** | 627.4 | 0.80**** | 0.84 | 0.73 | 0.73 |
| | (350.8) | (1098.5) | | | | | | |
| Importinⁿ | 4.5 | 11.6 | 2.6** | 0.7 | 0.71** | 0.68 | 0.81 | 0.30 |
| | (0.2) | (3.6) | | | | | | |
| ITGB1ⁿ | 32.4 | 15.3 | 0.5 | 13.5 | 0.46 | 0.71 | 0.43 | 0.30 |
| | (11.7) | (9.7) | | | | | | |
| Lysozymeⁿ | 145.7 | 303.6 | 2.1**** | 27.1 | 0.81**** | 0.71 | 0.89 | 0.73 |
| | (18.4) | (90.8) | | | | | | |
| M2-PKⁿ | 16.7 | 13.6 | 0.8 | 6.8 | 0.61 | 0.61 | 0.62 | 0.27 |
| | (5.7) | (8.5) | | | | | | |
| MMP-1^{p} | 1 | 16861.9 | 16861.9**** | 63.1 | 0.89**** | 1.00 | 0.78 | 0.83 |
| | (1) | (1754.2) | | | | | | |
| MMP-12ⁿ | 0.2 | 7.1 | 38.5**** | 0.1 | 0.80**** | 0.84 | 0.76 | 0.77 |
| | (0.1) | (0.8) | | | | | | |
| MMP-9^{p} | 31065.8 | 580512.7 | 18.7**** | 16931.4 | 0.85**** | 0.87 | 0.76 | 0.81 |
| | (2429.0) | (101655.7) | | | | | | |
| Properdin^{p} | 147.8 | 32692.8 | 221.1**** | 1211.6 | 0.89**** | 0.97 | 0.78 | 0.83 |
| | (1) | (7098.4) | | | | | | |
| Proteinase 3^{p} | 663.9 | 798.5 | 1.2**** | 85.9 | 0.81**** | 0.77 | 0.81 | 0.70 |
| | (49.3) | (271.5) | | | | | | |
| S100A12ⁿ | 170.5 | 288.7 | 1.7 | 132.8 | 0.51 | 0.87 | 0.19 | 0.22 |
| | (1) | (1) | | | | | | |
| S100A4ⁿ | 2.5 | 11.8 | 4.7 | 1.2 | 0.55 | 0.77 | 0.49 | 0.41 |
| | (0.9) | (1.1) | | | | | | |
| SOD1^{p} | 87977.1 | 138999.9 | 1.6ⁿ | 31671.7 | 0.64ⁿ | 0.65 | 0.65 | 0.30 |
| | (22695.1) | (42063.7) | | | | | | |
| SPARC^{p} | 6.9 | 16.6 | 2.4 | 8.7 | 0.64ⁿ | 0.94 | 0.38 | 0.46 |
| | (2.7) | (4.4) | | | | | | |
| TXD12ⁿ | 367.4 | 648.5 | 1.8 | 195.5 | 0.57 | 0.81 | 0.49 | 0.49 |
| | (149.2) | (173.1) | | | | | | |

In Table 2, biomarker protein units (normalized to creatinine) are as follows: n=ng/mg, p=pg/mg. Statistical significance was analyzed by Mann-Whitney U test comparing BC to UC. Sensitivity^{0.8} depicts the sensitivity at a fixed specificity value of 0.8. Indicated are the statistical significance p-values *p<0.05, **p<0.01, ***p<0.001, ****p<0.0001, comparing BC to UC.

### Example 2: Bladder Cancer Diagnostic Performance of Urine D-Dimer Without Creatinine Normalization

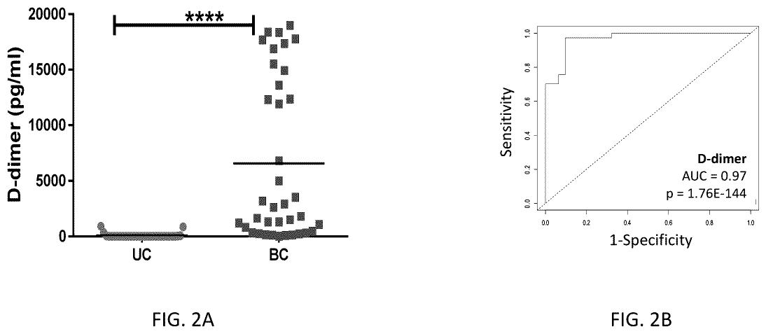

The discriminatory potential of urine D-dimer without creatinine normalization was assessed. As shown in FIGS. 2A and 2B, urine D-dimer (without creatinine normalization) was discriminatory between UC and BC, with an ROC AUC value of 0.97 (FIGS. 2A and 2B). Dot plot in FIG. 2A depicts urine D-dimer levels without creatinine normalization. Tested samples include 31 UC and 37 BC. Non-creatinine normalized urine protein levels are displayed. UC is represented by a circle and BC is represented by a square. The asterisks indicate the level of significance between the groups: ****p<0.0001. FIG. 2B depicts ROC-AUC plots generated for urine D-dimer in its ability to discriminate BC from UC, without creatinine normalization. AUC values and p-values are listed. If the AUC is close to 1, the protein has a higher discriminatory potential to distinguish between the two groups.

### Example 3: Bladder Cancer Diagnostic Performance of Urine D-Dimer With Creatinine Normalization

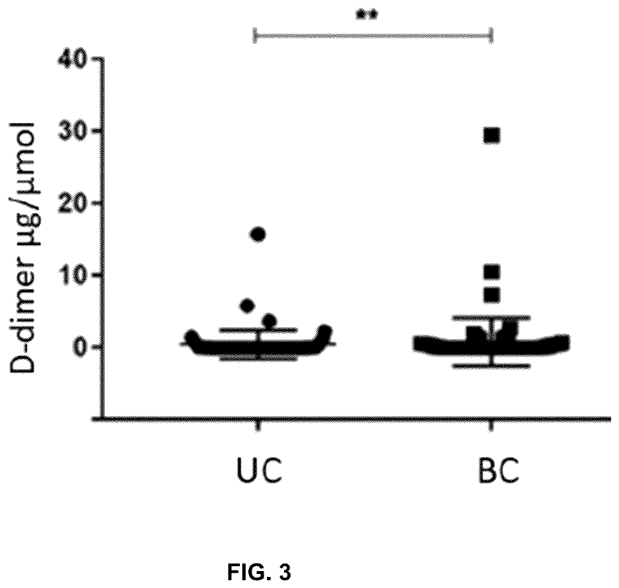

The diagnostic performance of urine D-dimer was assessed in a second cohort comprised of Chinese BC patients. As shown in FIG. 3, urine D-dimer was significantly higher in BC patients, compared to UC controls. Dot plot depicts urine D-dimer in a second validation cohort of Chinese ethnicity, comprised of BC patients (N=91) and UC (N=77) The UC subjects included 18 with kidney cancer, 50 with renal tract stones, and the rest with renal cyst, hamartoma, etc. Creatinine normalized protein values are shown for each group. UC is represented by a circle and BC is represented by a square. The asterisks indicate the level of significance between the groups: **p<0.01.

A subject who was previously diagnosed with and treated for bladder cancer is monitored for recurrence of bladder cancer according to the methods of the present disclosure. At a follow-up urology clinic visit, a urine sample is obtained from the subject and a level of D-dimer is measured in (assayed by ELISA or LFA). In response to the urine test being positive for D-dimer (i.e., a D-dimer level above a predetermined threshold), the patient undergoes a bladder biopsy (via a cystoscopy) to confirm the diagnosis of bladder cancer recurrence. In response to the urine test being negative for D-dimer (i.e., a D-dimer level at or below a predetermined threshold), the patient will simply be reviewed at the subsequent follow-up visit. The method provided herein saves the majority of patients from unnecessary invasive/expensive procedures, often with sub-par diagnostic profiles, leading to overall savings to the patient and overall healthcare and also reduce the incidence of cystoscopy-related side-effects. Early detection of bladder cancer can lead to decreased patient morbidity and mortality.

In patients with hematuria, it would be helpful to identify who needs invasive cystoscopic evaluation. Given that urine proteins are easily measurable and are compatible with point-of-care monitoring, a quick urine test could dramatically impact triage and workflow in urology outpatient clinics. Likewise, in bladder cancer surveillance, a reliable urine biomarker can help determine if cancer (such as carcinoma in situ) was missed or to avoid cystoscopy in marker-negative patients. Similarly, urine biomarkers that can reliably distinguish MIBC from NMIBC can inform a healthcare professional as to who has the more aggressive disease. When used as a routine point-of-care test (either at home or at outpatient visits), these urinary biomarkers may facilitate earlier identification of aggressive disease and design of tailored therapy.

As part of this work, >1000 urine proteins were screened for urine biomarker candidates in BC, using an aptamer-based screen. Systems biology analysis implicated molecular functions related to the extracellular matrix, collagen, integrin, heparin, and transmembrane tyrosine kinase signaling in BC susceptibility, with HNF4A and NFKB1 being key regulators. STEM analysis of the dysregulated pathways implicated a functional role for the immune system, complement, and interleukins in BC disease progression. Several urine proteins (D-dimer, Apolipoprotein A1, MMP-1, Properdin, Calgranulin B) significantly discriminate BC from UC with AUC values from 0.85 to 0.96 (p-value<0.0001). Urine D-dimer was able to discriminate BC from UC with 96% accuracy (sensitivity=95%; specificity=90%). As a single biomarker, urine D-dimer outperforms current FDA-approved biomarkers and competing biomarkers in the research literature as a sensitive biomarker for BC detection.

Embodiments of the disclosure include multi-marker panels and methods of using them for either a comparison of BC and UC or for a comparison of MIBC and NMIBC. These multi-marker panels can include two or more of urine D-dimer, MMP-1, Apolipoprotein A1, Proteinase 3, Apolipoprotein L1, IL-8, Ficolin-3, and Properdin. Certain embodiments include a multi-marker panel containing Apolipoprotein A1, D-dimer, and IL-8. Certain embodiments include a 5-marker panel containing urine D-dimer, MMP-1, Apolipoprotein A1, Proteinase 3, and Apolipoprotein L1. One such embodiment for the BC vs UC comparison includes this 5-marker panel with an AUC of 0.95, a sensitivity value of 0.89, and a specificity value of 0.87. Certain embodiments include a 5-marker panel containing urine IL-8, Ficolin-3, Apolipoprotein L1, Properdin, and Proteinase 3. One such embodiment for the MIBC vs NMIBC comparison includes this 5-marker panel with an AUC of 0.98, with a sensitivity of 0.79 and a specificity of 0.95. Three proteins in this panel (IL-8, Proteinase 3, Apolipoprotein L1) also ranked among the best single-marker performers, based on their individual AUC values. Another embodiment for the MIBC vs NMIBC comparison includes a multi-marker panel containing two or more of the following eight urine proteins: urine Apolipoprotein L1, complement C2, Endocan, Fibronectin, IgA, IL-8, MMP-12, and Proteinase 3. Their AUC values ranged from 0.75 to 0.99. Urine IL-8 was best at discriminating these two groups (AUC=0.99, sensitivity =100%; specificity=93%). As shown in Table 3, Urine IgA also outperformed other markers, with an AUC of 89%, a sensitivity of 86%, and a specificity of 87%. Of particular note, urine IgA exhibited the highest specificity of 80% for MIBC, at 80% sensitivity, outperforming IL-8.

The ability of 30 ELISA-validated urine proteins to discriminate bladder cancer patients by their disease stage own in Table 3. Thirty urinary proteins were assayed by ELISA, using the UTSW cohort, comprised of 7 MIBC and 30 NMIBC subjects. The ELISA results for MIBC vs NMIBC are displayed. Biomarker protein units (normalized to creatinine) are as follows: n=ng/mg, p=pg/mg. Specificity^{0.8} depicts the specificity at a fixed sensitivity of 0.8. Indicated are the statistical significance p-values as determined by a Mann-Whitney U-test *p<0.05; **p<0.01; ***p<0.001; ****p<0.0001.

TABLE 3
Results of the 30 ELISAs for the MIBC vs NMIBC stage comparisons.

| | NMIBC | MIBC | | | | | | | |
|---|---|---|---|---|---|---|---|---|---|
| | (mean/ | (mean/ | Fold | | | | Specificity | Sensitivity | |
| Protein | median) | medium) | Change | Cut Off | AUC | AUC CI | (CI) | (CI) | Sensitivity^{0.8} |
| 14-3-3 | 36.3 | 14.3 | 0.4 | 10.4 | 0.44 | 0.22-0.66 | 0.71 | 0.47(0.28-0.66) | 0.20 |
| sigma^{p} | (12.5) | (12.9) | | | | | (0.29-0.96) | | |
| α2 Macro- | 27502.0 | 25301.5 | 0.9 | 6418.3 | 0.72** | 0.55-0.89 | 1(0.59-1) | 0.53(0.34-0.72) | 0.60 |
| globulinⁿ | (4954.6) | (14389.6) | | | | | | | |
| α-synucleinⁿ | 4.6 | 1.5 | 0.3 | 0.4 | 0.55 | 0.33-0.77 | 1(0.59-1) | 0.37(0.20-0.56) | 0.37 |
| | (0.9) | (0.6) | | | | | | | |
| Apolipo- | 265.9 | 166.9 | 0.6 | 144.1 | 0.51 | 0.29-0.74 | 0.57 | 0.6(0.41-0.77) | 0.30 |
| protein | (118.0) | (144.1) | | | | | (0.18-0.90) | | |
| A1ⁿ | | | | | | | | | |
| Apolipo- | 359.2 | 1715.7 | 4.8** | 272.7 | 0.81*** | 0.64-0.98 | 0.86(0.42-1) | 0.67(0.47-0.83) | 0.67 |
| protein | (116.9) | (550.6) | | | | | | | |
| L1ⁿ | | | | | | | | | |
| C2ⁿ | 166.8 | 2050.0 | 12.3** | 796.3 | 0.85*** | 0.66-1 | 0.71 | 0.93(0.78-0.99) | 0.50 |
| | (26.1) | (1529.0) | | | | | (0.29-0.96) | | |
| Calgranulin | 98065.4 | 164828.2 | 1.7 | 62392.2 | 0.67 | 0.45-0.88 | 0.86(0.42-1) | 0.6(0.41-0.77) | 0.60 |
| Bⁿ | (38551.5) | (81775.8) | | | | | | | |
| CLU^{p} | 333363.0 | 338817.3 | 1 | 157994.9 | 0.61 | 0.40-0.82 | 0.86(0.42-1) | 0.47(0.28-0.66) | 0.47 |
| | (173919.1) | (235125.1) | | | | | | | |
| D-dimer^{p} | 13414.3 | 14193.7 | 1.1 | 4301.2 | 0.59 | 0.34-0.84 | 0.71 | 0.53(0.34-0.72) | 0.27 |
| | (3881.8) | (8111.01) | | | | | (0.29-0.96) | | |
| Elastaseⁿ | 36.2 | 51.6 | 1.4 | 28.6 | 0.66 | 0.42-0.90 | 0.57 | 0.8(0.61-0.92) | 0.47 |
| | (5.3) | (28.6) | | | | | (0.18-0.90) | | |
| Endocan^{p} | 470.6 | 3980.9 | 8.5* | 51.9 | 0.81*** | 0.64-0.99 | 0.71 | 0.8(0.61-0.92) | 0.60 |
| | (11.0) | (990.5) | | | | | (0.29-0.96) | | |
| Ficolin-3ⁿ | 349.5 | 493.0 | 1.4 | 626.6 | 0.46 | 0.16-0.75 | 0.29 | 0.9(0.74-0.98) | 0.00 |
| | (169.2) | (127.8) | | | | | (0.04-0.71) | | |
| Fibronectinⁿ | 713.4 | 2406.3 | 3.4** | 417 | 0.87**** | 0.75-0.99 | 1(0.59-1) | 0.67(0.47-0.83) | 0.80 |
| | (260.0) | (2226.4) | | | | | | | |
| HCC-1^{p} | 1924.1 | 9608.5 | 5 | 12685.9 | 0.6 | 0.34-0.86 | 0.29 | 0.93(0.78-0.99) | 0.28 |
| | (7.0) | (41.0) | | | | | (0.04-0.71) | | |
| IgAⁿ | 2175.0 | 5096.7 | 2.3*** | 3546.7 | 0.89**** | 0.78-1 | 0.86 | 0.87(0.69-0.96) | 0.80 |
| | (1182.7) | (4671.6) | | | | | (0.42-0.1) | | |
| IL-8^{p} | 1789.2 | 11863.1 | 6.6**** | 6824.8 | 0.99**** | 0.96-1 | 1(0.59-1) | 0.93(0.78(0.99) | 0.28 |
| | (824.5) | (9786.4) | | | | | | | |
| Importinⁿ | 10.7 | 15.9 | 1.5 | 6.8 | 0.72 | 0.50-0.94 | 0.71 | 0.8(0.61-0.92) | 0.63 |
| | (3.0) | (7.3) | | | | | (0.29-0.96) | | |
| ITGB1ⁿ | 16.5 | 10.8 | 0.6 | 4.8 | 0.47 | 0.27-0.66 | 1(0.59-1) | 0.23(0.10(0.42) | 0.27 |
| | (9.8) | (8.4) | | | | | | | |
| Lysozymeⁿ | 311.9 | 268.6 | 0.9 | 207.4 | 0.73* | 0.51-0.95 | 0.86(0.42-1) | 0.73(0.54-0.88) | 0.73 |
| | (77.6) | (230.9) | | | | | | | |
| M2-PKⁿ | 13.2 | 15.3 | 1.2 | 15.2 | 0.54 | 0.27-0.80 | 0.43 | 0.8(0.61-0.92) | 0.20 |
| | (9.0) | (6.8) | | | | | (0.10-0.82) | | |
| MMP-1^{p} | 11582.3 | 39489.2 | 3.4 | 1239.4 | 0.56 | 0.33-0.79 | 0.86(0.42-1) | (0.47 | 0.47 |
| | (1945.2) | (1754.1) | | | | | | (0.28-0.66) | |
| MMP-12ⁿ | 2.4 | 27.3 | 11.5* | 1.1 | 0.75** | 0.56-0.93 | 0.86(0.42-1) | 0.6(0.41-0.77) | 0.60 |
| | (0.6) | (1.8) | | | | | | | |
| MMP-9^{p} | 571808.7 | 617815.5 | 1.1 | 160857.6 | 0.67 | 0.43-0.90 | 0.86(0.42-1) | 0.67(0.47-0.83) | 0.67 |
| | (58493.6) | (233576.7) | | | | | | | |
| Properdin^{p} | 31064.9 | 39669.7 | 1.3 | 38589.3 | 0.55 | 0.30-0.81 | 0.43 | 0.8(0.61-0.92) | 0.13 |
| | (7425.5) | (6359.0) | | | | | (0.10-0.82) | | |
| Proteinase | 552.2 | 1854.2 | 3.4** | 773.2 | 0.84**** | 0.68-1 | 0.86(0.42-1) | 077(0.58-0.90) | 0.77 |
| 3^{p} | (232.6) | (1402.6) | | | | | | | |
| S100A12ⁿ | 314.2 | 179.4 | 0.6 | 1250 | 0.46 | 0.29-0.63 | 0.14(0-0.58) | 0.93(0.78-0.99) | 0.18 |
| | (1.0) | (1.0) | | | | | | | |
| S100A4ⁿ | 14.1 | 1.6 | 0.1 | 0.7 | 0.62 | 0.39-0.85 | 0.86(0.42-1) | 0.43(0.26-0.63) | 0.43 |
| | (1.0) | (1.3) | | | | | | | |
| SOD1^{p} | 143150.4 | 121211.7 | 0.8 | 8245.7 | 0.49 | 0.24-0.73 | 1(0.59-1) | 0.1(0.02-0.27) | 0.20 |
| | (43048.9) | (39984.9) | | | | | | | |
| SPARC^{p} | 12.5 | 34.4 | 2.8 | 8.7 | 0.69 | 0.43-0.94 | 0.71 | 0.7(0.51-0.85) | 0.30 |
| | (4.0) | (11.3) | | | | | (0.29-0.96) | | |
| TXD12ⁿ | 576.5 | 956.9 | 1.7 | 200 | 0.62 | 0.36-0.88 | 0.71 | 0.6(0.41-0.77) | 0.33 |
| | (163.4) | (280.3) | | | | | (0.29-0.96) | | |

Other objects, features and advantages of the disclosure will become apparent from the foregoing figures, detailed description, and examples. It should be understood, however, that the figures, detailed description, and examples, while indicating specific embodiments of the disclosure, are given by way of illustration only and are not meant to be limiting. Additionally, it is contemplated that changes and modifications within the spirit and scope of the disclosure will become apparent to those skilled in the art from the detailed description. In further embodiments, features from specific embodiments may be combined with features from other embodiments. For example, features from one embodiment may be combined with features from any of the other embodiments. In further embodiments, additional features may be added to the specific embodiments described herein.

What is claimed is:

1. A method for identifying a subject for an invasive testing for bladder cancer, the method comprising:
obtaining a first urine sample from the subject;
assessing an elevated level of D-dimer in a urine sample obtained from the subject as compared to a predetermined threshold level;
determining that the subject is at increased risk of having bladder cancer based on the elevated level of D-dimer in the urine sample; and
performing an invasive testing for the bladder cancer on the subject.

2. The method of claim 1, wherein the subject has had bladder cancer, and the assessing is for bladder cancer recurrence.

3. The method of claim 1, wherein the invasive testing for bladder cancer comprises one or more of cystoscopy, biopsy, and transurethral resection.

4. The method of claim 1, further comprising:
assessing an elevated level of one or more of MMP-1, Apolipoprotein A1, Proteinase 3, and Apolipoprotein L1 in the urine sample obtained from the subject as compared to predetermined threshold levels of MMP-1, Apolipoprotein A1, Proteinase 3, or Apolipoprotein L1; and
determining that the subject is at increased risk of having bladder cancer based on the elevated levels of the one or more of MMP-1, Apolipoprotein A1, Proteinase 3, and Apolipoprotein L1 in the urine sample.

5. The method of claim 1, further comprising:
assessing an elevated level of one or more of Apolipoprotein A1 and IL-8 in the urine sample obtained from the subject as compared to predetermined threshold levels of Apolipoprotein A1 or IL-8; and
determining that the subject is at increased risk of having bladder cancer based on the elevated levels of the one or more of Apolipoprotein A1 and IL-8 in the urine sample.

6. The method of claim 1, further comprising:
conducting urine cytology or assessing a level of nuclear matrix protein 22 in the urine sample obtained from the subject determined to be at an increased risk of having bladder cancer.

7. The method of claim 1, wherein the level of D-dimer in the urine sample is not normalized by a level of creatinine in the urine sample for the comparing with the predetermined threshold.

8. The method of claim 1, wherein the level of D-dimer in the urine sample is normalized with a level of creatinine in the urine sample for the comparing with the predetermined threshold.

9. A method for treating a subject for bladder cancer, the method comprising:
(a) assessing an increased level of D-dimer in a urine sample obtained from the subject as compared to a predetermined threshold level through an enzyme-linked immunosorbent assay (ELISA) or a lateral flow assay (LFA);
(b) in response to the level of D-dimer in the urine sample being above the predetermined threshold, identifying the subject as at increased risk of having bladder cancer;
(c) performing an invasive testing for the bladder cancer on the subject;
(d) determining that the subject has the bladder cancer based on results of the invasive testing; and
(e) treating the subject for the bladder cancer by one or more of surgical resection, radiation, chemotherapy, and hormone therapy.

10. The method of claim 9, wherein the subject has had bladder cancer, and is treated for bladder cancer recurrence.

11. The method of claim 9, wherein the invasive testing for bladder cancer comprises one or more of cystoscopy, biopsy, and transurethral resection.

12. The method of claim 9, further comprising:
assessing an elevated level of one or more of MMP-1, Apolipoprotein A1, Proteinase 3, and Apolipoprotein L1 in the urine sample obtained from the subject as compared to predetermined threshold levels of MMP-1, Apolipoprotein A1, Proteinase 3, or Apolipoprotein L1; and
determining that the subject is at increased risk of having bladder cancer based on the elevated levels of the one or more of MMP-1, Apolipoprotein A1, Proteinase 3, and Apolipoprotein L1in the urine sample.

13. The method of claim 9, further comprising:
assessing an elevated level of one or more of Apolipoprotein A1 and IL-8 in the urine sample obtained from the subject as compared to predetermined threshold levels of Apolipoprotein A1 or IL-8; and
determining that the subject is at increased risk of having bladder cancer based on the elevated levels of the one or more of Apolipoprotein A1 and IL-8 in the urine sample.

14. An assay system for assessing a level of D-dimer in a urine sample, for use in risk stratification for bladder cancer.

15. The assay system of claim 14, wherein the assay system is an enzyme-linked immunosorbent assay system.

16. The assay system of claim 14, comprising a lateral flow test strip.

17. The assay system of claim 14, comprising:
a capture reagent and a detection reagent that each bind to D-dimer;
a signal development element that can bind to the detection reagent;
a washing buffer;
a blocking buffer;
a calibration curve to relate a signal to the level of D-dimer; and/or an instruction for use.

18. The assay system of claim 14, wherein the assay system is a vertical flow assay.

19. The assay system of claim 14 for assessing levels of one or more of MMP-1, Apolipoprotein A1, Proteinase 3, and Apolipoprotein L1 in the urine sample, for use in risk stratification for bladder cancer.

20. The assay system of claim 14 for assessing levels of one or more of Apolipoprotein A1 and IL-8 in a urine sample, for use in risk stratification for bladder cancer.
